SEQUENCE LISTING (11 sequences)

SEQ ID NO: 1 - DNA, 51 bp, Homo sapiens
  misc_feature (26)..(26): SNP rs17024091; n is c or t
tatgagaaaatacatacatggtattnagcaccaatacttgatttcctatat

SEQ ID NO: 2 - DNA, 51 bp, Homo sapiens
  misc_feature (26)..(26): SNP rs2046542; n is a or g
aaaaaagatagtgggttggtcagtgntttcttacggctgctctcacagatt

SEQ ID NO: 3 - DNA, 51 bp, Homo sapiens
  misc_feature (26)..(26): SNP rs1386286; n is a or g
aaaaatgtgagcaggatgggaaaatncggcaaatgatggtcacatattctc

SEQ ID NO: 4 - DNA, 51 bp, Homo sapiens
  misc_feature (26)..(26): SNP rs3097662; n is c or t
ctagatcacatagacacccattattncatccagttgtttattttccccacc

SEQ ID NO: 5 - DNA, 51 bp, Homo sapiens
  misc_feature (26)..(26): SNP rs3117221; n is a or g
aatcctctagaccctaaactcagtgngggaccctcaccatccgtgtgcggc

SEQ ID NO: 6 - DNA, 51 bp, Homo sapiens
  misc_feature (26)..(26): SNP rs7759943; n is a or g
ttgttcttataatgggatactatacngcagttaaatgaattatagacatat

SEQ ID NO: 7 - DNA, 51 bp, Homo sapiens
  misc_feature (26)..(26): SNP rs1333934; n is g or t
ttcaatcaggaaactttctgtagganattgaccttggtctgtaaaggaaag

SEQ ID NO: 8 - DNA, 51 bp, Homo sapiens
  misc_feature (26)..(26): SNP rs13347411; n is a or g
tatattgttactttataacagtttcngatgcagcaaggcaatattatctct

SEQ ID NO: 9 - DNA, 51 bp, Homo sapiens
  misc_feature (26)..(26): SNP rs11651242; n is a, g or t
gtcactcagcctctctgagttccccncaataagtattcatgaaggacttac

SEQ ID NO: 10 - DNA, 51 bp, Homo sapiens
  misc_feature (26)..(26): SNP rs8067378; n is a or g
aaaaaatatttgtaacgttataaatngggaaaaacgtttatatcactgcca

SEQ ID NO: 11 - DNA, 51 bp, Homo sapiens
  misc_feature (26)..(26): SNP rs4282438; n is g or t
acaggttaagtatccatactccaagnaaatttggagagcatttcccagaga

(19) United States
(12) Patent Application Publication
(10) Pub. No.: US 2018/0112276 A1
(43) Pub. Date: Apr. 26, 2018

(54) METHODS AND KITS FOR DIAGNOSING OR ASSESSING THE RISK OF CERVICAL CANCER

(71) Applicant: Chang Gung Memorial Hospital, Linkou, Taoyuan City (TW)

(72) Inventors: CHYONG-HUEY LAI, Taoyuan City (TW); Angel Chao, Taoyuan City (TW); Chiao-Yun Lin, Taoyuan City (TW)

(21) Appl. No.: 15/792,096

(22) Filed: Oct. 24, 2017

Related U.S. Application Data
(60) Provisional application No. 62/413,088, filed on Oct. 26, 2016.

(51) Int. Cl. C12Q 1/68

(52) U.S. Cl. CPC C12Q 1/6886; C12Q 2600/118; C12Q 2600/156

(57) ABSTRACT

Kits for diagnosing or assessing the risk of developing cervical cancer in a subject, containing an agent, such as a primer, for detecting at least one single nucleotide polymorphism (SNP) are provided. Also provided are methods for diagnosing or assessing the risk of developing cervical cancer in a subject, comprising the detection of at least one SNP.

### CROSS-REFERENCES TO RELATED APPLICATION

This application claims the benefit of priority from U.S. Provisional Application No. 62/413,088, filed Oct. 26, 2016, the entire content of which is incorporated herein by reference.

### BACKGROUND OF THE INVENTION

The subject matter discussed in the background section should not be assumed to be prior art merely as a result of its mention in the background section. Similarly, a problem and the understanding of the causes of a problem mentioned in the background section should not be assumed to have been previously recognized in the prior art. The subject matter in the background section may merely represents different approaches, which in and of themselves may also be inventions.

Human papillomavirus (HPV) testing is widely used for cervical cancer screening. Women with vaginal HPV infection and normal cervical cytology are more likely to develop cervical cancer and/or cervical intraepithelial neoplasia (CIN) compared to women without vaginal HIV infection and normal cervical cytology. The diagnosis of HPV infection causes substantial anxiety in women as the risk of cervical cancer increases. However, apart from the viral infection, there are host risk factors that are associated with cervical cancer development.

The identification of the host risk factor is important for assessing the risk of developing cervical cancer, as well as alleviating the anxiety in women with vaginal HPV infection.

There is an unmet need for an economical and accurate laboratory diagnostic test for predicting the risk of cervical cancer and/or cervical intraepithelial neoplasia (CIN) development and the present invention satisfy this and other needs.

### BRIEF SUMMARY OF THE INVENTION

In one embodiment, the present invention discloses methods for diagnosing or assessing the risk of developing cervical cancer or CIN in a subject, comprising the steps of detecting at least one single nucleotide polymorphism (SNP) selected from rs17024091 (SEQ ID NO:1), rs2046542 (SEQ ID NO:2), rs1386286 (SEQ ID NO:3), rs3097662 (SEQ ID NO:4), rs3117221 (SEQ ID NO: 5), rs7759943 (SEQ ID NO:6), rs1333934 (SEQ ID NO:7), rs13347411 (SEQ ID NO:8), or rs11651242 (SEQ ID NO:9) in the sample of the subject, wherein the presence of at least one SNP is indicative of an increased risk for developing or having cervical cancer or CIN in the subject.

In another embodiment, the present invention discloses methods for diagnosing or assessing the risk of developing cervical cancer or CIN in a subject, comprising the steps of detecting at least two SNPs selected from rs8067378 (SEQ ID NO:10), rs4282438 (SEQ ID NO:11), or rs1386286 (SEQ ID NO:3) in the sample of the subject, wherein the presence of at least two SNPs is indicative of an increased risk for developing or having cervical cancer or CIN in the subject.

The present invention also discloses kits for diagnosing or assessing the risk of developing cervical cancer or CIN in a subject, comprising an agent for detecting at least one SNP selected from rs17024091, rs2046542, rs1386286, rs3097662, rs3117221, rs7759943, rs1333934, rs13347411 or rs11651242 in the sample of a subject.

The present invention further provides kits for diagnosing or assessing the risk of developing cervical cancer in a subject, comprising an agent for detecting at least two of the following SNPs: rs8067378, rs4282438, or rs1386286 in the sample of a subject.

In one embodiment, the kit described herein further comprises a label indicates that the agent is for detecting at least one SNP for assessing the risk of cervical cancer/CIN development or diagnosing cervical cancer/CIN.

The present invention also provides a set of SNPs for diagnosing or assessing the risk of developing cervical cancer/CIN, wherein the SNPs are selected from the group consisting of rs17024091, rs2046542, rs1386286, rs3097662, rs3117221, rs7759943, rs1333934, rs13347411, rs11651242, the combination of rs8067378 and rs4282438, the combination of rs8067378 and rs1386286, the combination of rs4282438 and rs1386286, and the combination of rs8067378, rs4282438, and rs1386286. Methods for diagnosing or assessing the risk of developing cervical cancer in a subject, comprising the step of detecting at least one SNP from the SNP set described above are provided. Also provided is a microarray for diagnosing or assessing the risk of developing cervical cancer or CIN, wherein the microarray comprises a set of polynucleotide to hybridize with the polynucleotide of at least one of the following SNP: rs17024091, rs2046542, rs1386286, rs3097662, rs3117221, rs7759943, rs1333934, rs13347411, rs11651242, the combination of rs8067378 and rs4282438, the combination of rs8067378 and rs1386286, the combination of rs4282438 and rs1386286, or the combination of rs8067378, rs4282438, and rs1386286. A kit for diagnosing or assessing the risk of developing cervical cancer or CIN comprising the microarray described herein is also provided.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

### BRIEF DESCRIPTION OF THE DRAWINGS

Illustrative embodiments of the present invention are described in detail below with reference to the following Figures:

FIG. 1 is flow charts illustrating the study design of the discovery cohort, verification cohort and prediction cohort of the Genome-Wide Association Study (GWAS) in Example 1.

FIG. 2 is a Manhattan plot, illustrating the SNPs associated with cervical intraepithelial neoplasia grade 2 or higher (CIN2⁺) based on Genome-Wide association study of Example 1.

FIG. 3 is a graph showing the cumulative incidence of cervical cancer progression in patients with no or one risk SNP (≤1 SNPs) and patients with more than one SNP (>1 SNPs).

### DETAILED DESCRIPTION OF THE INVENTION

As used herein, the articles “a” and “an” refer to one or more than one (i.e., at least one) of the grammatical object of the article.

The term “subject” may refer to a vertebrate suspected of having cervical cancer. Subjects include warm-blooded animals, such as mammals, such as a primate, and, more preferably, a human. Non-human primates are subjects as well. The tem′ subject includes domesticated animals, such as cats, dogs, etc., livestock (for example, cattle, horses, pigs, sheep, goats, etc.) and laboratory animals (for example, mouse, rabbit, rat, gerbil, guinea pig, etc.). In an embodiment, the subject has vaginal Human papillomavirus (HPV) infection.

All numbers herein may be understood as modified by “about.” As used herein, the term “about,” when referring to a measurable value, is meant to encompass variations of ±10%, preferably ±5%, more preferably ±1%, and even more preferably ±0.1% from the specified value, as such variations are appropriate to percentage of homology unless otherwise specified.

A multiple single nucleotide polymorphisms (SNPs) for assessing the risk of developing cervical cancer/CIN or diagnosing cervical cancer according to an embodiment of the present invention includes at least one polynucleotide sequences of SEQ ID NOS: 1 to 11.

TABLE 1

| Gene Bank accession | Polynucleotide |
|---|---|
| of SNP in NCBI | Containing SNP |
| rs17024091 | SEQ ID NO: 1 |
| rs2046542 | SEQ ID NO: 2 |
| rs1386286 | SEQ ID NO: 3 |
| rs3097662 | SEQ ID NO: 4 |
| rs3117221 | SEQ ID NO: 5 |
| rs7759943 | SEQ ID NO: 6 |
| rs1333934 | SEQ ID NO: 7 |
| rs13347411 | SEQ ID NO: 8 |
| rs11651242 | SEQ ID NO: 9 |
| rs8067378 | SEQ ID NO: 10 |
| rs4282438 | (SEQ ID NO: 11) |

The GenBank accession No. of an SNP in the National Center for Biotechnology Information (NCBI) database indicates a sequence and a position of the SNP. Those skilled in the art may easily identify the sequence and the position of the SNP using the GenBank accession No. The specific sequences corresponding to the rs No. of the SNP registered in NCBI may change over time. It is obvious to those skilled in the art that the sequences are within the scope of the present invention, even if the corresponding rs number changes. The nucleotide sequences of SEQ ID NOS: 1 to 11 are polymorphic sequences. A polymorphic sequence is a polynucleotide sequence including a polymorphic site representing SNP. The polynucleotide sequences can be DNA or RNA.

### Methods for Diagnosing or Assessing the Risk of Developing Cervical Cancer/CIN

The diagnosing method includes isolating DNA from a sample of a subject, determining a base sequence at a polymorphic site of the DNA, and judging that the subject has cervical cancer/CIN or has a high incidence/probability of cervical cancer/CIN when the base sequence includes at least one SNP selected from SEQ ID NO:1 to SEQ ID NO:9.

In certain embodiments, methods for diagnosing or assessing the risk of developing cervical cancer or CIN in a subject are provided, comprising the steps of detecting at least two SNPs selected from rs8067378, rs4282438 or rs1386286 in the sample of the subject, wherein the presence of at least two SNPs is indicative of cervical cancer/CIN or having an increased risk for developing cervical cancer or CIN in the subject. In an exemplary embodiment, the method comprises the step of detecting a first SNP at rs8067378 and a second SNP at rs4282438. In another embodiment, the method comprises the step of detecting a first SNP at rs8067378 and a second SNP at rs1386286. In yet another embodiment, the method comprises the step of detecting a first SNP at rs4282438 and a second SNP at rs1386286. In yet another embodiment, the method comprises the step of detecting a first SNP at rs4282438, a second SNP at rs1386286 and a third SNP at rs8067378.

The present invention is based, in part, on the identification of particular SNP in a test sample. Some embodiments of the present invention are directed to methods of assessing whether a subject has, or is at risk for developing, cervical cancer, comprising the identification of particular SNP in a test sample. Non limiting examples of the sample include DNA from peripheral white blood cells, serum, cell, tissue, or biopsy.

In some embodiments, molecular profiling can also including identifying a genetic variant, such as a mutation, polymorphism (such as a SNP), deletion, or insertion of a target. For example, identifying a SNP in a gene can be determined by microarray analysis, real-time PCR, or sequencing. Other methods disclosed herein can also be used to identify variants of one or more targets.

The polymorphisms can be detected by any available method, including amplification, hybridization to a probe or array, or the like, see B Sobrino et al, SNPs in forensic genetics: a review on SNP typing methodologies, Forensic Science International, Volume 154, Issues 2-3, 25 Nov. 2005, Pages 181-194. In one specific embodiment, SNP detection includes amplifying the polymorphism, linked locus or a sequence associated therewith (e.g., flanking sequences, transcribed sequences or the like) and detecting the resulting amplicon. For example, in one embodiment, amplifying includes a) admixing an amplification primer or amplification primer pair with a nucleic acid template isolated from the organism or biological sample. The primer or primer pair can be complementary or partially complementary to a region proximal to or including the polymorphism or linked locus, and are capable of initiating nucleic acid polymerization by a polymerase on the nucleic acid template. The primer or primer pair is extended in a DNA polymerization reaction comprising a polymerase and the template nucleic acid to generate the amplicon. In certain aspects, the amplicon is optionally detected by a process that includes hybridizing the amplicon to an array, digesting the amplicon with a restriction enzyme, or real-time PCR analysis. Optionally, the amplicon can be fully or partially sequenced, e.g., by hybridization. Typically, amplification can include performing a polymerase chain reaction (PCR), reverse transcriptase PCR (RT-PCR), or ligase chain reaction (LCR) using nucleic acid isolated from the organism or biological sample as a template in the PCR, RT-PCR, or LCR. Other technologies can be substituted for amplification, e.g., use of branched DNA (bDNA) probes.

These SNPs include SNPs associated with, e.g., the following genes: TMEM178: transmembrane protein 178A; RPL32P9: ribosomal protein L32 pseudogene 9; RING1: ring finger protein 1; VPS52: vacuolar protein sorting 52; NPAP1P4: nuclear pore associated protein 1 pseudogene 4; MTND2P9: mitochondrially encoded NADH: ubiquinone oxidoreductase core subunit 2 pseudogene 9; TNFRSF13B: tumor necrosis factor receptor superfamily, member 13B; IKZF3: IKAROS family zinc finger 3; ZPBP2: zona pellucida binding protein 2; GSDMB: gasdermin B; ORMDL3: ORM1-like 3; LRRC3C: leucine rich repeat containing 3C. Polymorphisms linked to these genes are, accordingly, also preferred SNPs that can be associated with cervical cancer polymorphisms.

Kits for Diagnosing or Assessing the Risk of Developing of Cervical Cancer and/or CIN

The present invention also provides kits for use in diagnosing or predicting the risk of developing cervical cancer and/or CIN. The kit comprises an agent for detecting at least one of the following SNPs located at rs17024091, rs2046542, rs1386286, rs3097662, rs3117221, rs7759943, rs1333934, rs13347411 or rs11651242.

The present invention further provides kits for assessing the risk of developing cervical cancer in a subject, comprising an agent for detecting at least two of the following SNPs: rs8067378, rs4282438, and rs1386286. In an exemplary embodiment, the kit comprises a first agent for detecting a first SNP at rs8067378 and a second agent for detecting a second SNP at rs4282438. In another embodiment, the kit comprises a first agent for detecting a first SNP at rs8067378 and a second agent for detecting a second SNP at rs1386286. In yet another embodiment, the kit comprises a first agent for detecting a first SNP at rs4282438 and a second agent for detecting a second SNP at rs1386286. In yet another embodiment, the kit comprises a first agent for detecting a first SNP at rs4282438, a second agent for detecting a second SNP at rs1386286 and a third agent for detecting a third SNP at rs8067378.

In one embodiment, the kit further comprises an instruction for using the agent to diagnose or assess the risk of developing cervical cancer or CIN. The agent can be an agent known in the art for detecting SNP.

Non-limiting examples of the agent include a primer set for isolating and amplifying DNA or a hybridization probe capable of detecting at least one SNP. The term “primer” refers to an oligonucleotide used in a polymerase chain reaction (PCR) reaction. The appropriate primer set may be easily designed by those skilled in the art with reference to the sequences according to an embodiment of the present invention.

In some embodiments, use of a microarray is desirable. A microarray is a microscopic, ordered array of nucleic acids, proteins, small molecules, cells or other substances that enables parallel analysis of complex biochemical samples. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

A microarray for cervical cancer diagnosis according to certain embodiments of the present invention includes allele-specific oligonucleotide (ASO) probes (i.e., polynucleotide hybridized with the polynucleotide of SEQ ID NOS:1-11). The ASO probes may be immobilized on a substrate coated with an active group selected among amino-silane, poly-L-lysine and aldehyde. Also, the substrate may be composed of a silicon wafer, glass, quartz, metal or plastic. The method of immobilizing the polynucleotide on the substrate may be either micropipetting using piezoelectric or a method using a pin-shaped spotter

A kit for cervical cancer diagnosis/assessing the risk of developing cervical cancer according to an embodiment of the present invention includes the microarray described herein.

Embodiments of the present invention are illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated. Some of the procedures are described below for illustrative purpose.

### Example 1: Genome-Wide Association Study (GWAS)

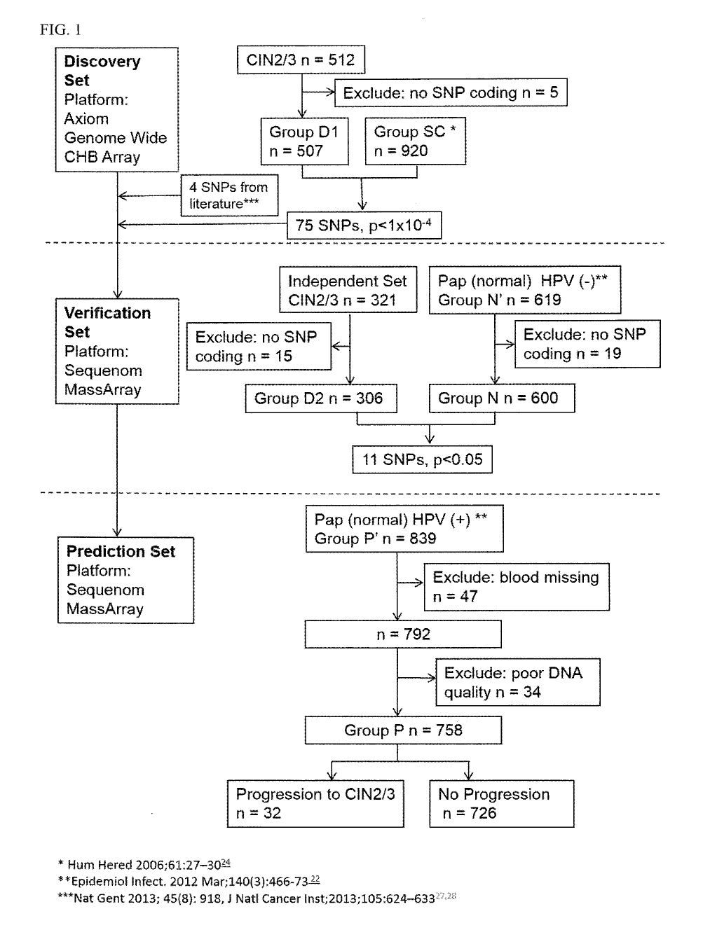

As illustrated in FIG. 1, a genome-wide association study (GWAS) of 642,832 single nucleotide polymorphisms (SNPs) by Affymetrix Axiom™ Genome-Wide Human Arrays was conducted on 507 cases with histological diagnosis of CIN2⁺ (group D1) versus 920 female controls (group C) as a discovery set.

The identified 75 CIN2⁺-associated SNPs from GWAS and 4 cervical cancer related SNPs from the literature were verified in an independent verification cohort as shown in FIG. 1. Group N [n=600] were HPV-negative/normal cytology women from a population-based cervical cytology and HPV co-test study whereas group D2 [n=306] are women with CIN2⁺.

In the prediction cohort (FIG. 1), subjects with HPV-positive/normal (group P, n=758) were followed up and served as the prediction cohort. The predictive validity was analyzed by logistic regression and receiver operating characteristic (ROC) curve analysis.

### Results

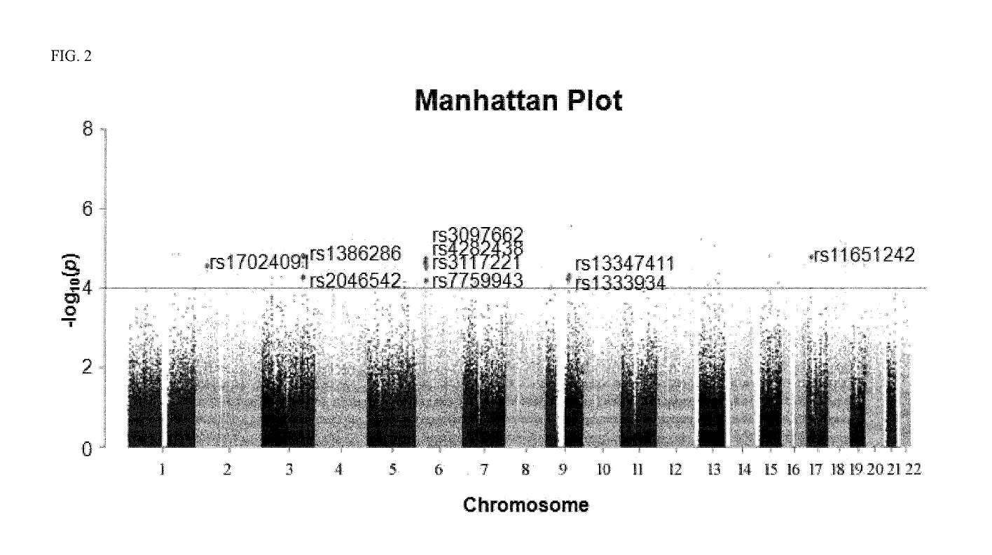

Thirty-two individuals of the group P progressed to CIN2+(median follow-up: 56.5 months, range 30.7-119.4). Ten out of seventy-five SNPs with trend test p values <10⁻⁴ identified from the discovery cohort along with rs8067378 reported from literature were validated in the verification set and approved to be associated with progression to CIN2⁺ (FIG. 2 and Table 2).

TABLE 2
SNPs identified from discovery cohort and validated in the verification cohort that are
associated with the risk of developing CIN²⁺ or cervical cancer.

| | | | | Minor | Minor | | | |
|---|---|---|---|---|---|---|---|---|
| | | | | Allele | Allele | | | |
| | | Physical | Major/ | Frequency | Frequency | | Trend | |
| dbSNP | | Position | Minor | in Case (N = | in Control | Risk | Test | |
| rsID | Chromosome | (bp) | Allele | 507) | (N = 920) | Allele | P-value | Nearby Gene |
| rs17024091 | 2 | 39847169 | T/C | 0.1578 | 0.2241 | T | 3.67 × 10⁻⁵ | TMEM178 |
| rs2046542 | 3 | 149832485 | C/T | 0.2875 | 0.3617 | C | 6.92 × 10⁻⁵ | LOC730021, RPL32P9 |
| rs1386286 | 3 | 149843853 | G/A | 0.2747 | 0.3513 | G | 2.24 × 10⁻⁵ | LOC730021, RPL32P9 |
| rs3097662 | 6 | 33020777 | T/C | 0.1907 | 0.1317 | C | 2.71 × 10⁻⁵ | HLA-DPA1, HLA- |
| | | | | | | | | DPB1, HLA-DPB2 |
| rs3117221 | 6 | 33061947 | T/C | 0.4772 | 0.3967 | C | 3.42 × 10⁻⁵ | HLA-DPA1, HLA- |
| | | | | | | | | DPB1, HLA-DPB2 |
| rs4282438 | 6 | 33072172 | T/G | 0.3901 | 0.4707 | T | 3.23 × 10⁻⁵ | HLA-DPA1, HLA- |
| | | | | | | | | DPB1, HLA-DPB2 |
| rs7759943 | 6 | 33195154 | G/A | 0.1028 | 0.062 | A | 9.92 × 10⁻⁵ | RING1, VPS52 |
| rs1333934 | 9 | 82935097 | A/C | 0.4448 | 0.37 | C | 7.58 × 10⁻⁵ | NPAP1P4 |
| rs13347411 | 9 | 83205419 | A/G | 0.3587 | 0.4354 | A | 7.32 × 10⁻⁵ | MTND2P9 |
| rs11651242 | 17 | 16854546 | T/G | 0.1755 | 0.2448 | T | 2.14 × 10⁻⁵ | TNFRSF13B |
| rs8067378* | 17 | 38051348 | A/G | 0.2931 | 0.263 | G | 0.0873 | IKZF3, ZPBP2, GSDMB, |
| | | | | | | | | ORMDL3, LRRC3C |

A risk-predictive SNP panel of rs8067378, rs4282438, and rs1386286 was generated from the discovery and verification set combining HPV types and tested in group P (sensitivity 0.844, negative predictive value 0.986, ROC 0.725) (Table 3).

TABLE 3
Distribution of risk-alleles and observed progression to
CIN2⁺ in group P stratified by HPV types

| | HPV high-risk^{a} | | | HPV low-risk^{b} | | |
|---|---|---|---|---|---|---|
| | | No. of | | | No. of | |
| Risk SNP(s) | N | Progression (%) | | N | Progression (%) | |
| 0 SNP | 6 | 0 | (0) | 5 | 0 | (0) |
| 1 SNP | | | | | | |
| (a1) rs8067378, | 4 | 0 | (0) | 3 | 0 | (0) |
| 1 risk allele | | | | | | |
| (a2) rs8067378, | 0 | 0 | (0) | 1 | 0 | (0) |
| 2 risk alleles | | | | | | |
| (b1) rs4282438, | 19 | 0 | (0) | 15 | 0 | (0) |
| 1 risk allele | | | | | | |
| (b2) rs4282438, | 6 | 0 | (0) | 7 | 0 | (0) |
| 2 risk alleles | | | | | | |
| (c1) rs1386286, | 38 | 1 | (2.6) | 27 | 0 | (0) |
| 1 risk allele | | | | | | |
| (c2) rs1386286, | 24 | 1 | (4.2) | 11 | 0 | (0) |
| 2 risk alleles | | | | | | |
| 2 SNPs | | | | | | |
| (a1) + (b1) | 13 | 1 | (7.7) | 7 | 0 | (0) |
| (a1) + (b2) | 5 | 0 | (0) | 4 | 0 | (0) |
| (a1) + (c1) | 23 | 0 | (0) | 9 | 0 | (0) |
| (a1) + (c2) | 20 | 1 | (5.0) | 13 | 0 | (0) |
| (a2) + (b1) | 0 | 0 | | 1 | 0 | (0) |
| (a2) + (b2) | 2 | 0 | (0) | 0 | 0 | (0) |
| (a2) + (c1) | 2 | 0 | (0) | 0 | 0 | (0) |
| (a2) + (c2) | 1 | 0 | (0) | 2 | 0 | (0) |
| (b1) + (c1) | 43 | 3 | (7.0) | 34 | 0 | (0) |
| (b1) + (c2) | 48 | 5 | (10.4) | 46 | 3 | (6.5) |
| (b2) + (c1) | 27 | 3 | (11.1) | 22 | 0 | (0) |
| (b2) + (c2) | 31 | 3 | (9.7) | 30 | 0 | (0) |
| 3 SNPs | | | | | | |
| (a1) + (b1) + (c1) | 35 | 3 | (8.6) | 29 | 0 | (0) |
| (a1) + (b1) + (c2) | 29 | 3 | (10.3) | 24 | 1 | (4.2) |
| (a1) + (b2) + (c1) | 20 | 2 | (10.0) | 19 | 0 | (0) |
| (a1) + (b2) + (c2) | 15 | 2 | (13.3) | 11 | 0 | (0) |
| (a2) + (b1) + (c1) | 2 | 0 | (0) | 0 | 0 | (0) |
| (a2) + (b1) + (c2) | 6 | 0 | (0) | 3 | 0 | (0) |
| (a2) + (b2) + (c1) | 5 | 0 | (0) | 3 | 0 | (0) |
| (a2) + (b2) + (c2) | 5 | 0 | (0) | 3 | 0 | (0) |

^{a}HPV16, 18, 31, 33, 35, 39, 45, 51, 52, 56, 58, 59, 66, and 68.
^{b}HPV6, 11, 26, 32, 37, 42, 43, 44, 53, 54, 55, 61, 62, 67, 69, 70, 71, 72, 74, 81, 82, 83, 84, L1AE5

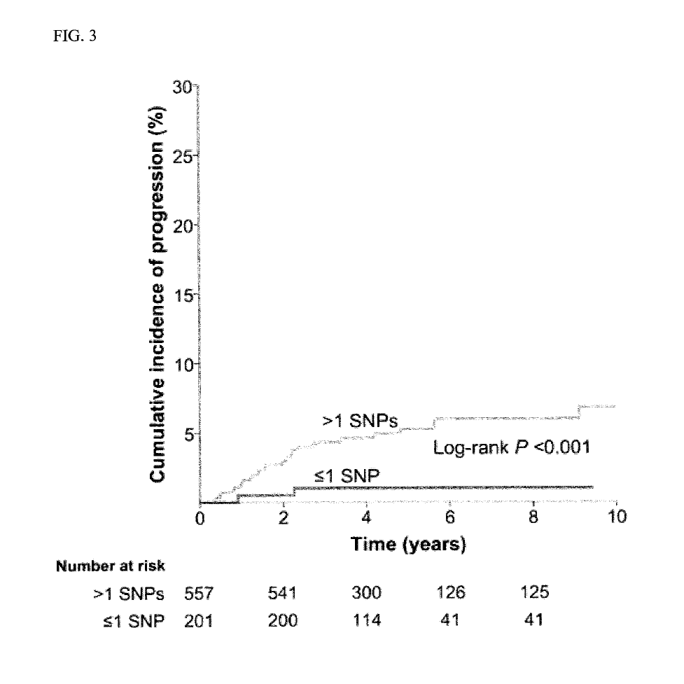

As illustrated in FIG. 3, women carried ≤1 risk-allele had significantly lower hazard ratio (0.21 [0.05−0.86], p=0.031) of cervical cancer progression than those with >1 risk-allele (1.7% for ≤1 risk-allele vs. 10.7% for >1 risk-allele).

What is claimed is:

1. A method for diagnosing or assessing the risk of developing cervical cancer in a subject, comprising the step of detecting at least one single nucleotide polymorphism (SNP) selected from rs17024091, rs2046542, rs1386286, rs3097662, rs3117221, rs7759943, rs1333934, rs13347411, rs11651242, a combination of rs8067378 and rs4282438, a combination of rs8067378 and rs1386286, a combination of rs4282438 and rs1386286, or a combination of rs8067378, rs4282438, and rs1386286 in the subject, wherein the presence of at least one of the SNPs is indicative of an increased risk for developing cervical cancer in the subject.

2. The method of claim 1, wherein the SNP is detected in a serum sample.

3. The method of claim 1, wherein the subject has vaginal human papilloma virus infection.

4. A kit for diagnosing or assessing the risk of developing cervical cancer, comprising:
an agent for detecting at least one single nucleotide polymorphism (SNP) selected from rs17024091, rs2046542, rs1386286, rs3097662, rs3117221, rs7759943, rs1333934, rs13347411, rs11651242, a combination of rs8067378 and rs4282438, a combination of rs8067378 and rs1386286, a combination of rs4282438 and rs1386286, or a combination of rs8067378, rs4282438, and rs1386286 in the sample of a subject.

5. The kit of claim 4, wherein the sample is DNA from peripheral white blood cells.

6. The kit of claim 4, wherein said agent is a primer.

7. The kit of claim 4, wherein the said agent is an allele-specific oligonucleotide (ASO) probes.

8. The kit of claim 4, further comprising a label indicates that the agent for detecting the risk of cervical cancer.
